(19) United States
(12) Patent Application Publication
(10) Pub. No.: US 2006/0200219 A1
(43) Pub. Date: Sep. 7, 2006

(54) Systems and methods for differentiating and/or identifying tissue regions innervated by targeted nerves for diagnostic and/or therapeutic purposes

(75) Inventors: Geoffrey B. Thrope, Shaker Heights, OH (US); Joseph J. Mrva, Euclid, OH (US); Robert B. Strother, Willoughby Hills, OH (US); Kenneth P. Rundle, Independence, OH (US)

Correspondence Address: RYAN KROMHOLZ & MANION, S.C., POST OFFICE BOX 26618, MILWAUKEE, WI, 53226, US

(73) Assignee: NDI Medical, LLC

(21) Appl. No.: 11/337,319

(22) Filed: Jan. 23, 2006

Related U.S. Application Data
(63) Continuation-in-part of application No. 11/099,848, filed on Apr. 6, 2005.
(60) Provisional application No. 60/657,277, filed on Mar. 1, 2005.

(51) Int. Cl. A61B 5/04; A61N 1/36; A61N 1/08; A61N 1/18

(52) U.S. Cl. 607/145; 607/2; 607/9; 607/48; 607/40; 600/509

(57) ABSTRACT

Systems and methods make possible the differentiation and identification of tissue regions locally innervated by targeted nerves. The systems and methods make it possible to access the nervous system at these localized regions for therapeutic benefit. For example, the systems and methods can be used to access parasympathetic nerves localized in fat pads on the surface of the heart.

### RELATED APPLICATION

This application is a continuation-in-part of co-pending U.S. patent application Ser. No. 11/099,848, filed Apr. 6, 2005 and entitled “Systems and Methods for Intra-Operative Stimulation,” which claims the benefit of U.S. Provisional Patent Application Ser. No. 60/657,277, filed Mar. 1, 2005, and entitled “Systems and Methods for Intra-Operative Stimulation,” which are incorporated herein by reference.

### FIELD OF THE INVENTION

The invention relates generally to systems and methods for differentiation and/or identification of tissue regions targeted for diagnostic or therapeutic purposes.

### BACKGROUND OF THE INVENTION

The autonomic nervous system governs the involuntary processes of the glands, large internal organs, cardiac muscle, and blood vessels. The autonomic nervous system as a whole exerts a continuous, local control over the function of many organs (such as the eye, lung, urinary bladder, and genitalia). The autonomic nervous system consists of the sympathetic and the parasympathetic systems.

The sympathetic system initiates a series of reactions, called “fight-or-flight” reactions, that prepare the body for activity. The heart rate increases, blood pressure rises, and breathing quickens. The amount of glucose in the blood rises, providing a reservoir of quick energy. The flow of blood to the skin and organs decreases, allowing more blood to flow to the heart and muscles.

The parasympathetic system generally functions in an opposite way, initiating responses associated with rest and energy conservation; its activation causes breathing to slow, salivation to increase, and the body to prepare for digestion.

It may be desirable for diagnostic and/or therapeutic reasons to differentiate and/or identify within a tissue region the presence of targeted sympathetic nerves and/or parasympathetic nerves.

### SUMMARY OF THE INVENTION

The invention provides devices, systems, and methods for differentiating and/or identifying tissue regions locally innervated by targeted nerves. The systems and methods make it possible to access the nervous system at these localized regions for diagnostic or therapeutic purposes.

One aspect of the invention provides a first device for generating and applying a stimulation current to tissue. The devices, systems, and methods also include a second device for sensing the presence or absence of an anticipated physiologic response to the application of the electrical stimulation current. The presence of the anticipated physiologic response indicates the innervation of targeted nerve fibers or branches within the tissue region. Once differentiated and identified, the targeted nerve fibers or branches can be manipulated to achieve desired diagnostic and/or therapeutic outcomes.

The devices, systems, and methods are well suited, e.g., for differentiating and/or identifying localized branches of the vagus nerve. The vagus nerve runs from the brain through the face and thorax to the abdomen. It is a mixed nerve that contains parasympathetic fibers. The vagus nerve has the most extensive distribution of the cranial nerves. Its pharyngeal and laryngeal branches transmit motor impulses to the pharynx and larynx; its cardiac branches act to slow the rate of heartbeat; its bronchial branch acts to constrict the bronchi; and its esophageal branches control involuntary muscles in the esophagus, stomach, gallbladder, pancreas, and small intestine, stimulating peristalsis and gastrointestinal secretions. Being able to differentiate and/or identify the presence of a branch of the vagus nerve within a given tissue region within the body makes possible the development and application of diverse diagnostic and/or therapeutic techniques for parasympathetic mediation of a diverse number of anatomic functions, e.g., in the digestive system, the respiratory system, or the heart.

For example, one aspect of the invention provides devices, systems, and methods that make possible the differentiation and identification of the epicardial fat pads on the surface of the heart, which are innervated by parasympathetic vagal nerve fibers. The devices, systems, and methods thereby make it possible to access the parasympathetic nervous system of the heart for therapeutic benefits, such as to control the ventricular rate or to provide physiologic control of the AV nodal rate.

Another aspect of the invention provides systems and methods for treating a heart comprising locating a fat pad region on a heart innervated by parasympathetic nerves using a first device for generating and applying a stimulation current, and then manipulating the parasympathetic nervous system of the heart in the region of the fat pad for diagnostic or therapeutic benefit.

Features and advantages of the inventions are set forth in the following Description and Drawings, as well as the appended description of technical features.

### BRIEF DESCRIPTION OF THE DRAWINGS

FIG. 1 is a diagrammatic view of a system for differentiating and/or identifying tissue regions locally innervated by targeted nerves.

FIG. 2A is side view of a device used in conjunction with the system shown in FIG. 1 for generating and applying a stimulation current to tissue in the region of the targeted nerve fiber or branch.

FIG. 2B is side view of an alternative embodiment of the device shown in FIG. 2A, and having separate amplitude and duration selection switches.

FIG. 3A is an enlarged view of one embodiment of a bipolar electrode array that the device shown in FIGS. 2A or 2B may carry at its distal end.

FIG. 3B is an enlarged view of an additional embodiment of a bipolar electrode array that the device shown in FIGS. 2A or 2B may carry at its distal end.

FIG. 3C is an enlarged view of an additional embodiment of a bipolar ring electrode array that the device shown in FIGS. 2A or 2B may carry at its distal end.

FIG. 4 is a representative view of a clinician manipulating the device shown in FIG. 2A in association with the system shown in FIG. 1.

FIG. 5 is an anatomic posterior view of a human heart, showing the location of fat pads innervated by parasympathetic nerves that, when accessed, can provide therapeutic benefits.

FIGS. 6 and 7 are diagrammatic views of use of the system shown in FIG. 1 for differentiating and/or identifying a fat pad tissue region that is locally innervated by parasympathetic nerves.

### DESCRIPTION OF PREFERRED EMBODIMENTS

I. The System

FIG. 1 shows a system 10 for differentiating and/or identifying within a tissue region TR the presence of a targeted nerve fiber or branch. The system 10 includes a first device 12 for generating and applying a stimulation current to tissue in the region TR of the targeted nerve fiber or branch. The system 10 also includes a second device 14 for sensing the presence or absence of an anticipated physiologic response to the application of the electrical stimulation current. The presence of the anticipated physiologic response differentiates and/or identifies within a tissue region TR the presence of a targeted nerve fiber or branch. Once differentiated and identified, the targeted nerve fiber or branch can be manipulated for desired diagnostic and/or therapeutic reasons.

A. The First Device

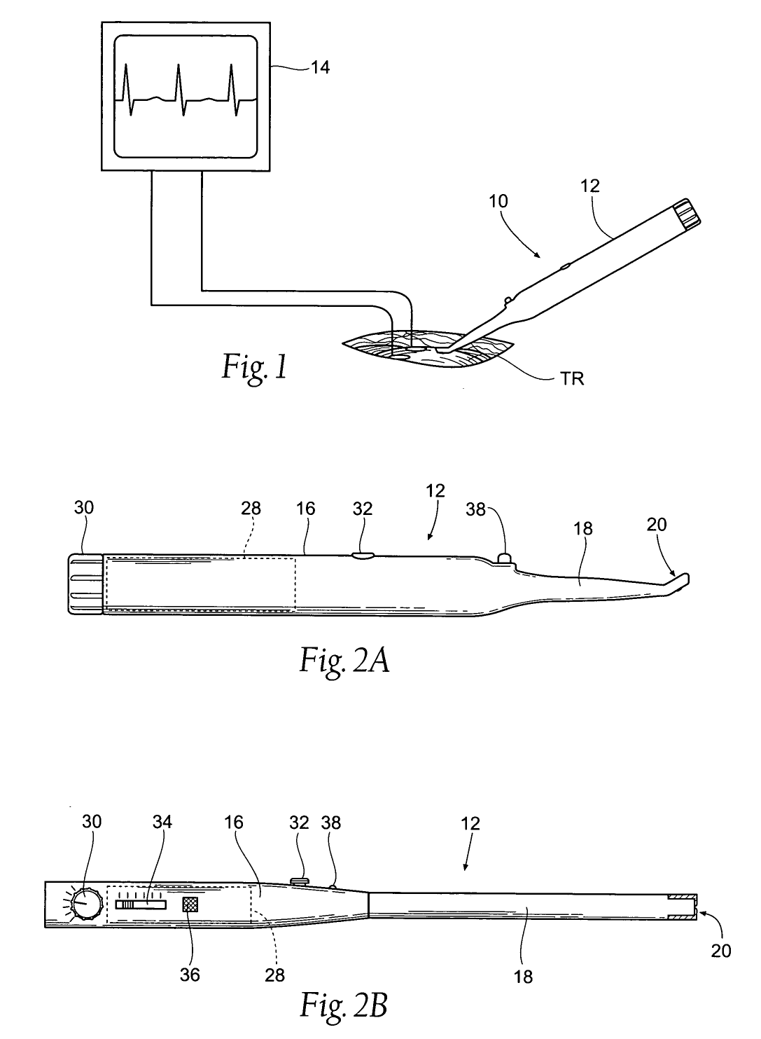

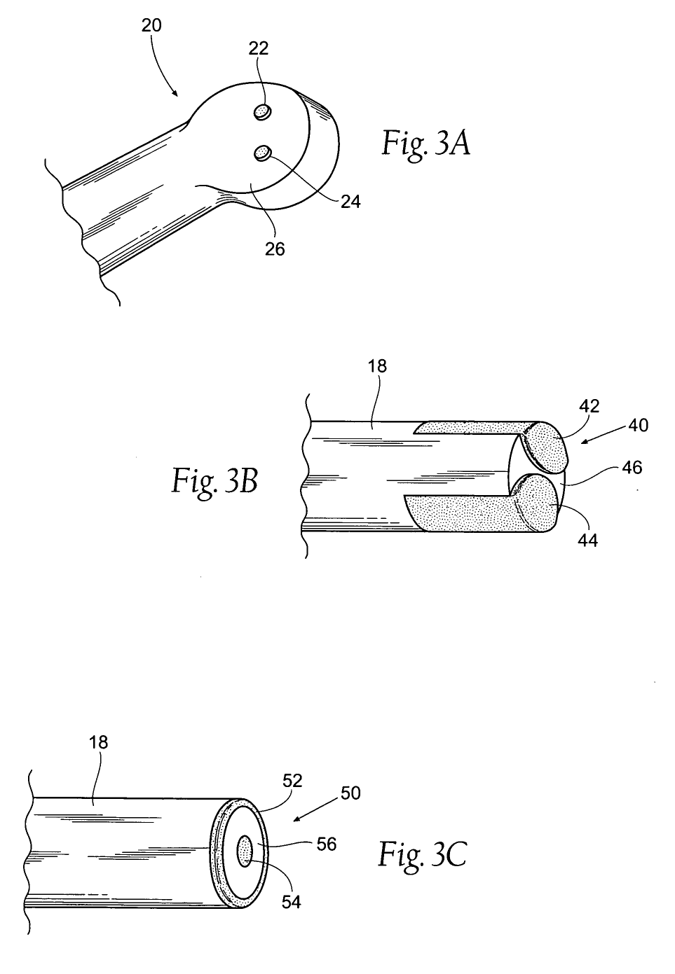

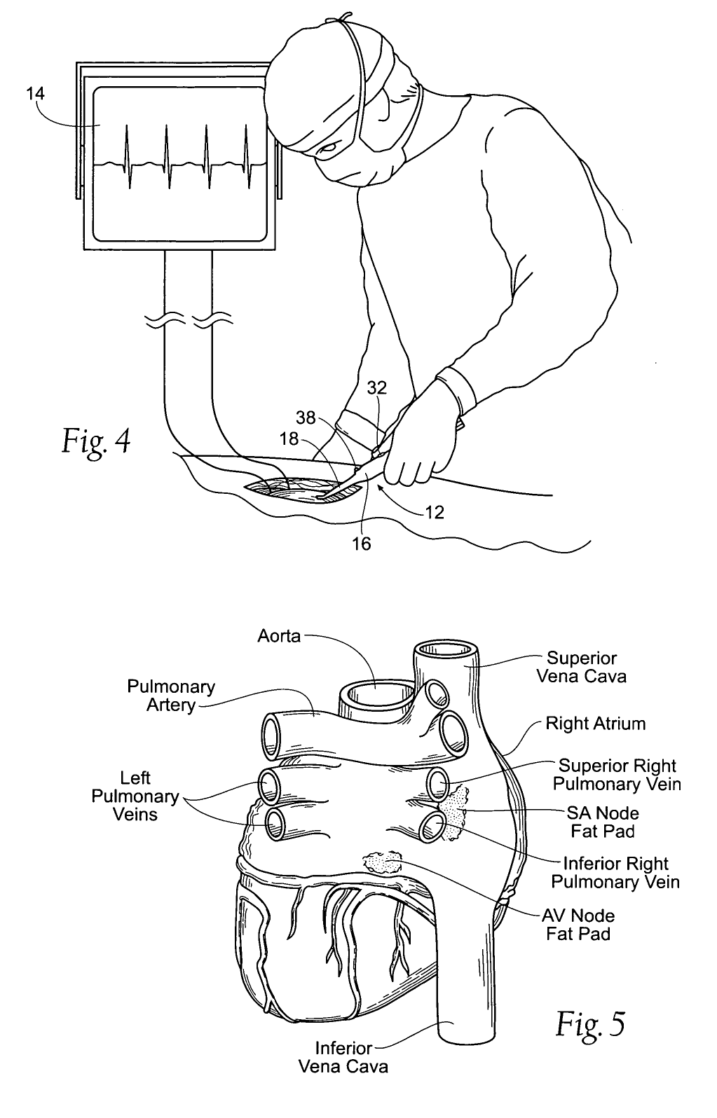

As FIGS. 2A to 4 show, the first device 12 includes a handle 16, which is preferably sized small enough to be held and used like a flashlight or screwdriver, allowing the thumb to push a button to control the application of stimulus current (see FIG. 4). The handle 16 carries an insulated probe 18. The probe 18 carries, at its distal end, an electrode assembly 20 (see FIG. 3A). The first device 12 is preferably a sterile, single use instrument.

In a representative embodiment, the handle 16 is cylindrical in shape and has a maximum diameter at its proximal end of about 25 mm. The handle 16 tapers from proximal end to distal end to a lesser diameter of about 10 mm. In a representative embodiment, the length of the handle 16 is about 17 cm.

In a representative embodiment, the probe 18 extends about 8 cm from the distal end of the handle 16 and includes an electrode assembly 20 at its distal end. In a representative embodiment, the probe 18 has a diameter of about 10 mm.

The electrode assembly 20 (see FIG. 3A) is sized and configured for accurate identification of tissue regions innervated by targeted nerves. The electrode assembly 20 may be configured to resemble something like a dental mirror and may have a diameter in the range of about 10 mm to about 15 mm. The assembly 20 may be somewhat offset (e.g., 10 degrees to 50 degrees), from the probe 18 to provide ease of use and a more ergonomic configuration. The electrode assembly 20 may comprise a bipolar array of two contacts 22 and 24 exposed on the distal face 26 of the probe 18. The contacts 22 and 24 may have a diameter in the range of about 1 (one) mm to about 3 mm and may project off the distal face by 1 (one) mm or less. The spacing between the contacts 22 and 24 on the distal face 26 may be about 1 (one) mm to about 4 mm. The edges of the contacts 22 and 24 are desirably rounded, so as not to injure tissue. The small area of the contacts 22 and 24 ensures a high current density that will stimulate nearby excitable tissue.

It is to be appreciated that other configures for an electrode assembly may be possible. For example, FIGS. 3B and 3C show two additional possible configurations. FIG. 3B shows an electrode assembly 40 having contacts 42 and 44 exposed on the distal face 46 of the probe 18. The contacts 42 and 44 are circumferentially spaced 180-degrees apart. As shown, the contacts 42 and 44 are exposed on the distal face 46 of the probe 18, each occupying about 90-degrees to about 95-degrees of the circumference of the distal face 46 of the probe 18. The contacts 42 and 44 also desirably extend proximally along the probe for about 5 mm, as well as project a short distance beyond the distal face 46 of the probe 18, e.g., 1 mm. Spacing between the contacts 42 and 44 on the distal face 46 may be about 1 (one) mm to about 4 mm. The edges of the contacts 42 and 44 are desirably rounded, so as not to injure tissue. FIG. 3C shows a ring electrode assembly having an outer contact 52 and an inner contact 54 exposed on the distal face 56 of the probe 18. The outer contact 52 may also extend proximally along the probe.

The contacts 22 and 24 (and their alternative embodiments) can comprise, e.g., stainless steel, silver, platinum, or platinum treated with platinum black. The probe 18 comprises, especially at its distal face 26, a plastic material that is preferably poorly wetted by blood, saline, and body fluids, so as to minimize the risk of passing current through the fluid pathway when direct tissue contact is not present. The probe 18 is insulated from the handle 16 using common insulating means (e.g., wire insulation, washers, gaskets, spacers, bushings, and the like).

Alternatively, a monopolar arrangement can be used. In this arrangement, a return electrode (or indifferent electrode) must be provided to provide an electrical path from the body back to the instrument. The return electrode may be placed on the surface of intact skin (e.g., surface electrodes, such as used for ECG monitoring during surgical procedures) or it might be needle-like and be placed in the surgical field or penetrate through intact skin.

An electrical stimulation control circuitry 28 is carried within the handle 16 (see FIGS. 2A and 2B). The control circuitry 28 generates a stimulation current which is applied through the contacts 22 and 24. The control circuitry 28 is powered by a primary battery (for single use applications) located within the handle 16. If the instrument is not intended for single use, the battery can be rechargeable.

The control circuitry 28 desirably includes an on-board, programmable microprocessor, which carries embedded code. The code expresses pre-programmed rules or algorithms for generating the desired electrical stimulation waveforms. In a representative embodiment, the stimulus frequency is 20 Hz, (although the frequency may be adjustable, e.g., 3 Hz to 100 Hz), and the waveform comprises a charge balanced biphasic waveform (i.e., no net DC current flow).

Other operating parameters of the control circuitry 28 can be regulated by controls conveniently carried on the handle 16.

In the illustrated embodiment (see FIG. 2A), stimulus amplitude and the stimulus pulse duration are adjusted by a rotary switch 30 or wheel near or on the proximal end of the handle 16. The rotary control switch 30 desirably has labeling to identify multiple setting options. For example, the first few settings may include different amplitudes each with the same fixed pulse duration. Additional settings may provide a range of selectable settings that include specific combinations of amplitudes and pulse durations. The rotary control switch 30 also desirably has detents that gives the clinician good tactile feedback when moving from one setting to the next. The range of stimulus settings labeled can comprise, e.g., OFF, STANDBY, 1.5 mA at 100 μsec, 3 mA at 100 μsec, 5 mA at 100 μsec, 5 mA at 300 μsec, and 10 mA at 500 μsec.

A momentary pushbutton 32, e.g., on the side of the housing 16, e.g., for access by a thumb, controls the delivery of the stimulation current through the contacts 22 and 24. The momentary pushbutton 32 allows the first device 12 to be controlled, e.g., stimulation current to be turned on and off, with only one hand. The stimulus current is delivered (at the amplitude/duration set by the rotary switch 30) through the contacts 22 and 24 only if the momentary pushbutton 32 is depressed. If the pushbutton 32 is not depressed, no stimulus current is delivered.

In an alternative embodiment (see FIG. 2B), the stimulus pulse duration may be regulated by an adjustable stepped slide switch 34 on the handle 16. Thus, if the momentary pushbutton 32 is depressed, stimulus current is applied at the regulated amplitude and regulated duration. If the pushbutton 32 is not depressed, no stimulus current is delivered. The slide switch 34 desirably has labeling to identify the pulse duration selected. The slide switch 34 also desirably has detents that gives the clinician good tactile feedback when moving from one pulse duration level to the next. The range of pulse duration settings labeled can comprise, e.g., OFF, 100 μsec, 300 μsec, or 500 μsec. The slide switch 34 could also have a STANDBY position labeled.

Alternatively, if the pulse duration slide switch 34 is not provided, and the pulse duration is not selected via the rotary control switch 30, the stimulus pulse durations can be fixed at a nominal selected duration, e.g., 250 μsec.

The control circuitry 28 desirably includes a light indication, i.e., a light emitting diode LED 38 on the handle, that provides various indications to the clinician. For example, the LED 38 may confirm battery status and stimulator ON/OFF states. Also desirably, the LED 38 may flash green when adequate stimulus is being delivered, and flash red when inadequate stimulus is delivered. In addition, the LED 38 may flash or illuminate only if the current actually delivered is within a desired percentage of the requested amplitude, e.g., within 25% of the requested value. The control circuitry 28 thereby provides reliable feedback to the clinician as to the requested delivery of stimulus current.

In an alternative embodiment, the control circuitry 28 may also generate an audio tone only when the stimulus current is being delivered. The tone is transmitted by an indicator 36 on the handle 16.

Through the use of different tones, colors, different flash rates, etc., the control circuitry 28 can allow the clinician to confirm that the probe is in contact with tissue, the instrument is turned ON, the battery has sufficient power, and that stimulus current is flowing. Thus the clinician has a much greater confidence that the failure to elicit a desired response is because of lack of viable nervous tissue near the tip of the probe rather than the failure of the return electrode connection or some other instrumentation problem.

B. The Second Device

The second device 14 can take various forms, depending upon the physiologic function of the targeted tissue region and the nature and character of the physiologic response anticipated due to the application of the electrical stimulation current by the first device 12.

For example, the electrical stimulation of parasympathetic nerves affecting a respiration activity causes breathing to slow. Therefore, when it is desired to differentiate and/or identify the presence or absence of parasympathetic nerves affecting a respiration activity, a reduction in the breathing rate can be used as the anticipated physiologic response. In this arrangement, the second device 14 can comprise an instrument that monitors breathing. The instrument can comprise, e.g., a chest position sensor and a spirometer box that monitor movements of the chest. The instrument can also comprise a breathing sensor, which is worn around the chest, such as a breathing (stretch) sensor or a stethograph. A decrease in breathing rate detected by the second device indicates that the first device is located at or near parasympathetic nerves.

As another example, the stimulation of parasympathetic nerves affecting heart function increases the resting potential and decreases the rate of diastolic depolarization. Under these circumstances the heart rate slows. Therefore, when it is desired to differentiate and/or identify the presence or absence of parasympathetic nerves affecting heart activity, the heart rate can be used as the anticipated physiologic response. In this arrangement, the second device 14 can comprise an electrocardiography (EKG) instrument.

As another example, the stimulation of parasympathetic nerves affecting digestion (e.g., during the cephalic phase of gastric secretion) mediates reflex gastric secretion. Therefore, when it is desired to differentiate and/or identify the presence or absence of parasympathetic nerves affecting stomach activity, the reduction in the secretion of gastric juice can be used as the anticipated physiologic response. In this arrangement, the second device 14 can comprise instrumentation that senses the secretion of gastric juice.

As another example, the second device 14 can comprise an electromyography (EMG) instrument. The EMG instrument measures nerve impulses within muscles. The EMG system includes electrodes that are placed in the muscles in the tissue region innervated with parasympathetic nerves, and the electronic responses to operation of the first device 12 can be observed using an instrument that displays movement of an electric current (e.g., an oscilloscope). As muscles contract, they emit a weak electrical signal that can be detected, amplified, and tracked as the anticipated physiologic response.

III. Use of the System

In use, the first device 12 is positioned in contact with tissue in a targeted tissue region TR. A clinician may operate the first device 12 with one hand to apply the stimulation current. The clinician's other hand can then be used to make adjustments to the stimulation current as necessary. The second device 14 monitors the physiologic response. The first device 12 is located and relocated (if necessary) until the monitored physiologic response indicated by the second device 14 matches or approximates the anticipated physiologic response. This indicates the presence of the targeted nerve fiber or branch, and the identified location may then be marked. A desired treatment regime can then be performed, e.g., to manipulate the parasympathetic nervous system for therapeutic benefit.

For example, it has been observed that the parasympathetic nervous system of the heart can be manipulated to coordinate cardiac conduction and/or function as relates to atrial fibrillation, without tissue ablation and without interrupting physiologic conduction. It is known that parasympathetic nerve fibers of the vagus nerve can be manipulated to affect atrial cycle length. It is also known that parasympathetic nerve fibers of the vagus nerve selectively innervate the epicardial antrioventricular (AV) node fat pad and the sinoatrial (SA) node fat pad (as FIG. 5 shows).

The system 10 makes possible, e.g., the differentiation and identification of the epicardial AV node fat pad on the surface of the heart, and thereby makes it possible to access the parasympathetic nervous system of the heart at this location for therapeutic benefit.

More particularly, the first device 12 of the system 10 makes possible the application highly localized electrical stimulation on the surface of the heart, while the second device 14 monitors heart rate. The clinician may start the application of the stimulus current at the lowest amplitude setting, and increase the amplitude setting as necessary. Adjustments may be necessary due to the physiological differences of tissue regions from patient to patient. The clinician may also start the application of the stimulus current at something other than the lowest amplitude setting after a visual inspection of the tissue region TR indicates that a higher initial setting may be necessary.

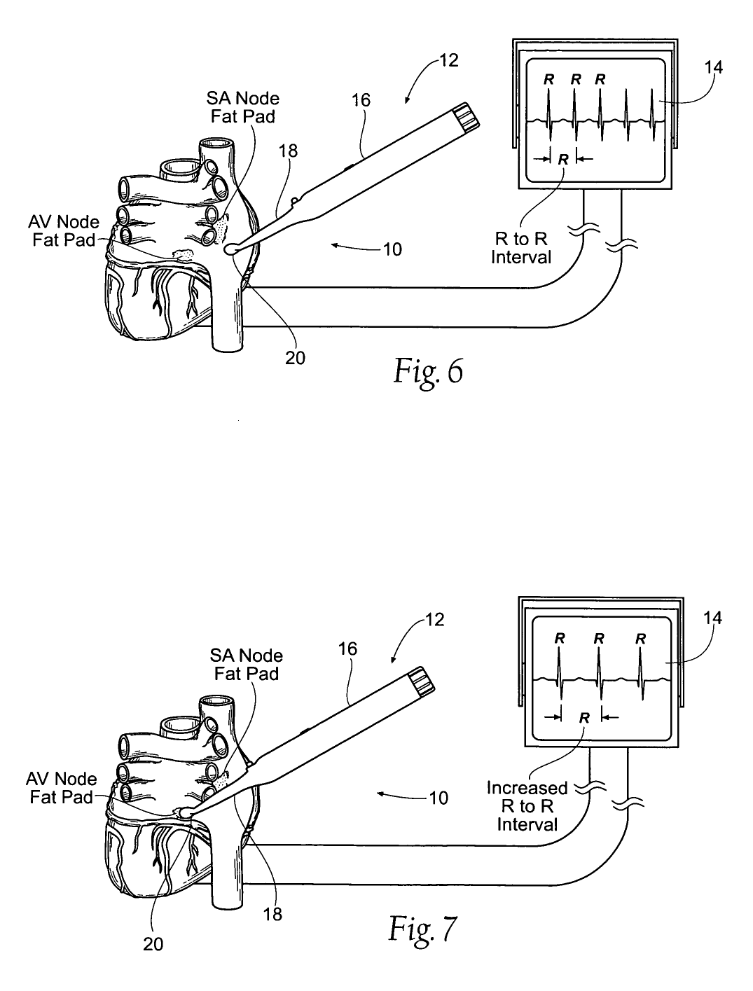

When the first device 12 is applying stimulation and is ultimately located at or near the region of the AV node fat pad (see FIG. 7), the heart rate (monitored by the second device 14, e.g., an EKG instrument) will decrease. An EKG instrument 14 will indicate a decrease in heart rate by an increase in the R-to-R interval observed on EKG (compare the R-to-R interval shown in FIG. 6 to the increased R-to-R interval shown in FIG. 7). The clinician may then stop the application of stimulation current to the tissue region, e.g., the identified AV node fat pad, and observe an increase in the heart rate returning to the original heart rate (a decrease in the R-to-R interval observed on EKG). The clinician may go through the steps of applying stimulation current, observing an increase of the R-to-R interval, stopping the application of stimulation current, and observing a decrease in the R-to-R interval, to confirm the accurate location of the targeted tissue region, e.g., the AV node fat pad. In this way, the system 10 allows a clinician to systematically and accurately locate the AV node fat pad (and other regions selectively innervated by parasympathetic nerves) on the surface of the heart.

Once located, the clinician may use the first device 12 to apply a die or other marker to maintain identification of the AV node fat pad. Alternatively, a separate applicator may be used to apply a die or other marker, or, the clinician may use visual skills along with their finger, for example, to maintain identification of the AV node fat pad. The clinician can then take steps to perturb the parasympathetic nervous system of the heart for therapeutic benefit. For example, by either electrical or non-electrical manipulation of the AV node fat pad located by the system 10, the clinician can treat or prevent uncontrolled atrial fibrillation or perform other desired therapies, or the clinician can apply closed-loop feed-back control algorithms that provide physiologic control of AV nodal rate.

Manipulation of the AV node fat pad located by the system 10 preserves physiologic conduction. With electrical manipulation, its beneficial effects can be turned on and turned off instantaneously, and without attenuation of effect. Manipulation of the AV node fat pad may provide a viable alternative to AV node ablation in the treatment of atrial fibrillation, which does not preserve physiologic conduction and instead consigns patients to pacemaker dependency.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

We claim:

1. A system for differentiating and/or identifying targeted tissue regions locally innervated by a targeted nerve comprising:
a hand held instrument including an electrode configuration for applying an electrical stimulation current to a tissue region, the hand held instrument including a handle, an electrical stimulation control circuitry carried within the handle, and at least one controller carried on the handle and coupled to the control circuitry for selectively altering at least one operating parameter of the control circuitry, and
a second device that indicates a physiologic response to the presence or absence of the electrical stimulation current.

2. A system according to claim 1
wherein the targeted nerve is the vagus nerve or its branches.

3. A system according to claim 1
wherein the targeted tissue regions are epicardial fat pads on the surface of the heart.

4. A system according to claim 1
wherein the second device comprises an electrocardiography (EKG) instrument.

5. A system according to claim 1
wherein the second device comprises an instrument that monitors breathing.

6. A system according to claim 1
wherein the second device comprises an instrument that senses the secretion of gastric juice.

7. A system according to claim 1
wherein the electrode configuration comprises a bipolar array of two contacts exposed on a distal face of a probe extending off of the handle.

8. A system according to claim 1
wherein the hand held instrument is a sterile, single use instrument.

9. A system according to claim 1
wherein the operating parameters include stimulation current amplitude and stimulation pulse duration.

10. A method for treating a heart comprising
locating a fat pad region on a heart innervated by parasympathetic nerves using a system as defined in claim 1, and
manipulating the parasympathetic nervous system of the heart in the region of the fat pad for diagnostic or therapeutic benefit.

11. A method according to claim 10
further including applying a die or marker to the fat pad region to memorialize the fat pad region.

12. A method according to claim 10
wherein locating further includes applying an electrical stimulation,
observing a physiologic response to the application of the electrical stimulation,
stopping the application of the electrical stimulation, and
observing a physiologic response to the stopping the application of the electrical stimulation.
